(19) United States
(12) Patent Application Publication
(10) Pub. No.: US 2026/0232744 A1
(43) Pub. Date: Aug. 13, 2026

(54) COMPOSITION

(71) Applicant: HONDA MOTOR CO., LTD., Tokyo (JP)

(72) Inventors: Nozomi SHIOBARA, Saitama (JP); Shohei KINOSHITA, Saitama (JP); Mizuho DOI, Saitama (JP); Minoru GOTO, Saitama (JP); Kenji MACHIDA, Saitama (JP)

(21) Appl. No.: 19/160,473

(22) PCT Filed: Feb. 2, 2024
(86) PCT No.: PCT/JP2024/003445

§ 371 Date: Aug. 28, 2025

(30) Foreign Application Priority Data
Mar. 2, 2023 (JP) 2023-031925

(51) Int. Cl. A61K 36/05; A61K 8/9722; A61P 39/06; A61Q 19/08

(52) U.S. Cl. CPC A61K 36/05; A61K 8/9722; A61P 39/06; A61Q 19/08

(57) ABSTRACT

Provided is a composition containing Chlamydomonas reinhardtii or an extract of Chlamydomonas reinhardtii, wherein the Chlamydomonas reinhardtii or the extract of the Chlamydomonas reinhardtii contains an anti-active oxygen substance.

### TECHNICAL FIELD

The present invention relates to a composition including Chlamydomonas reinhardtii or an extract of Chlamydomonas reinhardtii.

### BACKGROUND ART

Pharmaceutical products are being developed to ensure healthy lives and promote well-being for all people of all ages.

Cited Document 1 discloses J005 strain of the genus Chlorella that is capable of growing under dark culture conditions, has a high chlorophyll content and high active oxygen scavenging effect (Accession No. FERMP-21127).

On the other hand, Patent Document 2 discloses Honda DREAMO strain (Accession No. FERM BP-22306) as an example of the Chlamydomonas reinhardtii.

### CITATION LIST

### Patent Document

Patent Document 1: Japanese Unexamined Patent Application, Publication No. 2008-193995

Patent Document 2: PCT International Publication No. WO2017/217116

### DISCLOSURE OF THE INVENTION

### Problems to be Solved by the Invention

However, it is not known that the Chlamydomonas reinhardtii or extracts of Chlamydomonas reinhardtii function as an anti-active oxygen substance.

An object of the present invention is to provide a composition containing, as an active ingredient, Chlamydomonas reinhardtii or an extract of Chlamydomonas reinhardtii that functions as an anti-active oxygen substance.

### Means for Solving the Problems

(1) A composition including Chlamydomonas reinhardtii or an extract of Chlamydomonas reinhardtii, in which the Chlamydomonas reinhardtii or the extract of Chlamydomonas reinhardtii includes an anti-active oxygen substance.

(2) The composition as described in (1), in which the Chlamydomonas reinhardtii is a Honda DREAMO strain (Accession No. FERM BP-22306).

(3) The composition as described in (1) or (2), which is an anti-aging agent.

(4) The composition as described in any one of (1) to (3), in which the Chlamydomonas reinhardtii is a freshwater alga.

### Effects of the Invention

According to the present invention, it is possible to provide a composition containing, as an active ingredient, Chlamydomonas reinhardtii or an extract of Chlamydomonas reinhardtii that functions as an anti-active oxygen substance.

### PREFERRED MODE FOR CARRYING OUT THE INVENTION

Hereinafter, embodiments of the present invention will be described.

The composition of this embodiment includes Chlamydomonas reinhardtii or an extract of Chlamydomonas reinhardtii, and the Chlamydomonas reinhardtii or the extract of Chlamydomonas reinhardtii includes an anti-active oxygen substance.

Examples of the Chlamydomonas reinhardtii include, but are not particularly limited to, UTEX 90 strain and Honda DREAMO strain (Accession No. FERM BP-22306). Here, the UTEX 90 strain is the original strain of the Honda DREAMO strain.

Preferably, the Chlamydomonas reinhardtii is a freshwater alga. As a result, a large amount of culture can be performed on land.

The composition of the present embodiment can be applied to an anti-aging agent, because it contains, as an active ingredient, Chlamydomonas reinhardtii or an extract of Chlamydomonas reinhardtii that functions as an anti-active oxygen substance.

Administration forms of the anti-aging agents may be either oral administration (internal use) or parenteral administration (external use, injection).

Dosage forms of the anti-aging agents are not particularly limited, and examples thereof include solid preparations such as tablets, granules, powders, capsules, etc.; liquid preparations such as solutions, suspensions, emulsions, etc.; lyophilized preparations and the like.

The anti-aging agents can be manufactured using known methods.

The anti-aging agents may be applied to, for example, pharmaceutical products, quasi-pharmaceutical products, cosmetics, or foods.

The composition of the present embodiment is preferably used for humans, but may be used for animals other than humans.

A culture method of the Chlamydomonas reinhardtii is not particularly limited, and examples thereof include a static culture method, a shaking culture method, a deep culture method, and an aeration culture method.

The Chlamydomonas reinhardtii can be cultured in large quantities on land by culturing outdoors. A method of culturing the Chlamydomonas reinhardtii outdoors is not particularly limited, and examples thereof include a method of culturing the Chlamydomonas reinhardtii in an open pond system using a raceway bioreactor, a method of culturing the Chlamydomonas reinhardtii using a flat panel photobioreactor installed outdoors, and the like.

A medium used for culturing the Chlamydomonas reinhardtii is not particularly limited, and examples thereof include a TAP medium, a urea medium, an HSM medium, and a BG11 medium.

A temperature during culturing the Chlamydomonas reinhardtii is not particularly limited, but is, for example, 2° C. or higher and 38° C. or lower.

A light and dark cycle when culturing the Chlamydomonas reinhardtii is not particularly limited, and is, for example, a 24-hour cycle, in which a light phase is 6 hours or more and 24 hours or less.

A photosynthesis effective photon flux density in the light phase is not particularly limited, but is, for example, 50 μmol/m²/s or more and 2,000 μmol/m²/s or less.

A gas aeration amount per unit volume when culturing the Chlamydomonas reinhardtii by the aeration culture method is not particularly limited, but is, for example, 0.001 VVM or more and 10 VVM or less. It should be noted that VVM indicates how many times the volume ratio of the gas with respect to the medium is aerated in one minute.

Carbon dioxide is preferably supplied to the medium when culturing the Chlamydomonas reinhardtii by the aeration culture method. A concentration of carbon dioxide in the gas to be aerated is not particularly limited, but is, for example, 0.1% by volume or more and 10% by volume or less. A supply source of carbon dioxide is not particularly limited, and examples thereof include a gas discharged from a carbon dioxide cylinder, a factory, or the like, and concentrated carbon dioxide recovered from the atmosphere by direct air capture (DAC).

A method for recovering the cultured Chlamydomonas reinhardtii is not particularly limited, and examples thereof include a centrifugation method, a filtration method, or the like.

The recovered Chlamydomonas reinhardtii may be dried as necessary.

A method for drying the Chlamydomonas reinhardtii is not particularly limited, and examples thereof include a freeze drying method, a spray drying method, a hot air drying method, a sun drying method, and the like.

An extraction method used for extracting the Chlamydomonas reinhardtii is not particularly limited, and examples thereof include an ultrasonic extraction method.

A solvent used for extraction of the Chlamydomonas reinhardtii is not particularly limited, and examples thereof include lower alcohols such as methanol, ethanol, propyl alcohol, isopropyl alcohol, etc.; lower aliphatic ketones such as acetone, methyl ethyl ketone, etc.; polyhydric alcohols such as 1,3-butylene glycol, propylene glycol, glycerin, etc.; and hydrophilic organic solvents such as dimethyl sulfoxide (DMSO), etc. Among these, DMSO is preferable.

A method for recovering an extract solution of Chlamydomonas reinhardtii is not particularly limited, and examples thereof include a centrifugal separation method, a filtration method, etc.

The recovered extract of Chlamydomonas reinhardtii may be dried as necessary.

A method for drying the extract of Chlamydomonas reinhardtii is not particularly limited, and examples thereof include a freeze-drying method, etc.

### EXAMPLES

Hereinafter, examples of the present invention will be described, but the present invention is not limited to the examples.

### Example 1

### [Culture of Chlamydomonas Reinhardtii]

Using 2 L of a TAP medium, Honda DREAMO strain as the Chlamydomonas reinhardtii was statically cultured (seed culture) for 5 days under the culture conditions shown below.
Temperature: 25° C.
Light and Dark Cycle: 12 hours of light phase and 12 hours of dark phase
Photosynthesis effective photon flux density in the light phase: 60 to 80 μmol/m²/s
Light Source: white LED

Next, 500 mL of the seed culture product was centrifuged at 3000 rpm for 5 minutes, the culture supernatant was removed, and the seed-cultured Honda DREAMO strain was recovered.

Next, the recovered Honda DREAMO strain was statically cultured (pre-culture) for 7 days using 2 L of the TAP medium under the same culture conditions as in the seed culture.

Next, aeration culture of the pre-cultured Honda DREAMO strain was carried out for 7 days by using 5 L of a urea medium (main culture) under the same culture conditions as in the seed culture. The aeration conditions were as follows.
Gas aeration per unit volume: 0.15 VVM
Concentration of carbon dioxide in the gas: 5 vol %

Next, 5 L of the main culture product was centrifuged at 6,000 rpm for 10 minutes, the culture supernatant was removed, and the main cultured Honda DREAMO strain was recovered.

### [Extraction]

Approximately 20 ml of the recovered Honda DREAMO strain was sonicated with 100 mL of DMSO for 5 minutes using an ultrasonic homogenizer. At this time, ultrasonic extraction was performed on ice.

Next, a mixture of an extract solution and a residue was centrifuged at 3,000 rpm for 5 minutes, the residue was removed, and about 100 mL of the extract solution was recovered.

Next, the extract solution was freeze-dried to obtain 490.74 mg of the extract of Honda DREAMO strain.

### [ORAC Values]

Measurement showed that an ORAC (oxygen radical absorbance capacity) value of the Honda DREAMO strain extract was 280 μmol TE/g FW. Thus, it can be seen that the Honda DREAMO strain extract contained an anti-active oxygen substance, that is, the Honda DREAMO strain extract can function as an anti-active oxygen substance. Incidentally, the measurement of the ORAC value was outsourced to Japan Food Research Laboratories.

1. A composition comprising Chlamydomonas reinhardtit or an extract of Chlamydomonas reinhardtii,
wherein the Chlamydomonas reinhardtii or the extract of Chlamydomonas reinhardtii comprises an anti-active oxygen substance.

2. The composition according to claim 1, wherein the Chlamydomonas reinhardtii is a Honda DREAMO strain (Accession No. FERM BP-22306).

3. The composition according to claim 1, which is an anti-aging agent.

4. The composition according to claim 1, wherein the Chlamydomonas reinhardtii is a freshwater alga.
